(19) United States
(12) Patent Application Publication
(10) Pub. No.: US 2011/0160296 A1
(43) Pub. Date: Jun. 30, 2011

(54) PREPARATION OF 6-OXA-8ALPHA-STEROID ESTROGEN ANALOGUES - A NEW GROUP OF UNNATURAL ESTROGENS AND THEIR USE IN MEDICINE

(75) Inventors: Ulrich Pison, Berlin (DE); Alexander Grigorievich Shawa, St. Petersburg (RU); Svetlana Nikolaevna Morozkina, St. Petersburg (RU)

(73) Assignee: TOPASS GMBH, Berlin (DE)

(21) Appl. No.: 12/741,696

(22) PCT Filed: Nov. 10, 2008
(86) PCT No.: PCT/EP2008/009619

§ 371 Date: Mar. 11, 2011

(30) Foreign Application Priority Data
Nov. 9, 2007 (EP) 07075983.2

(51) Int. Cl. A61K 31/352; C07J 73/00; C07D 311/78; A61P 15/00; A61P 29/00; A61P 19/10; A61P 9/00; A61P 35/00; A61P 19/04; A61P 25/00; A61P 25/28; A61P 3/06; A61P 3/00

(52) U.S. Cl. 514/453; 549/383; 549/384

(57) ABSTRACT

The invention is related to the area of new 6-Oxa-8α-steroid estrogen analogues and the synthesis of these new biological active steroid estrogen analogues, namely, to the preparation of 6-oxa-8α-steroid estrogens and their use as estrogen receptor modulators. These new estrogen analogues are ligands for estrogen receptors and as such may be useful for the treatment and prevention of a variety of conditions related to estrogen functioning. These conditions include bone and cartilage disorders, increased levels of LDL cholesterol, cardiovascular diseases, impairment of cognitive function, cerebral degeneration disorders, endometriosis and other types of inflammation, the metabolic syndrome, and cancer, in particular of the breast, uterus and prostate.

The invention is related to the area of new 6-Oxa-8α-steroid estrogen analogues and the synthesis of these new biological active steroid estrogen analogues, namely, to the preparation of 6-oxa-8α-steroid estrogens and their use as estrogen receptor modulators. These new estrogen analogues are ligands for estrogen receptors and as such may be useful for the treatment and prevention of a variety of conditions related to estrogen functioning. These conditions include bone and cartilage disorders, increased levels of LDL cholesterol, cardiovascular diseases, impairment of cognitive function, cerebral degeneration disorders, endometriosis and other types of inflammation, the metabolic syndrome, and cancer, in particular of the breast, uterus and prostate.

### BACKGROUND OF THE INVENTION

Naturally occurring and synthetic estrogens have broad therapeutic utility, including relief of menopausal symptoms, treatment of breast and prostatic cancer, treatment of various types of inflammation, treatment of dysmenorrhea and dysfunctional uterine bleeding, treatment of osteoporosis, treatment of hirsutism and prevention of cardiovascular disease. Because estrogen is very therapeutically valuable, there is great interest in the synthesis and preparation of unnatural estrogen analogues or discovering other compounds that mimic estrogen-like behaviour in estrogen responsive tissues.

For example, estrogen-like compounds would be beneficial in the treatment and prevention of menopausal symptoms such as osteoporosis. Osteoporosis affects approximately 20 to 25 million post-menopausal women in the U.S. alone. It has been theorized that the rapid loss of bone mass in these women is due to the cessation of estrogen production in the ovaries. Since studies have shown that estrogen slows the reduction of bone mass due to osteoporosis, estrogen replacement therapy is a recognized treatment for postmenopausal osteoporosis.

In addition to bone mass, estrogen appears to have an effect on the biosynthesis of cholesterol and cardiovascular health. Statistically, the rate of occurrence of cardiovascular disease is roughly equal in postmenopausal women and men; however, premenopausal women have a much lower incidence of cardiovascular disease than men. Because postmenopausal women are estrogen deficient, it is believed that estrogen plays a beneficial role in preventing cardiovascular disease. The mechanism is not well understood, but evidence indicates that estrogen can upregulate the low density lipid (LDL) cholesterol receptors in the liver to remove excess cholesterol.

Postmenopausal women given estrogen replacement therapy experience a return of lipid levels to concentrations comparable to levels associated with the premenopausal state. Thus, estrogen replacement therapy could be an effective treatment for such disease. However, the side effects associated with long term estrogen use limit the use of this alternative.

Breast cancer and uterine cancer are other disease states that affect post-menopausal women. Anti-estrogen compounds, such as tamoxifen, have commonly been used as chemotherapy to treat breast cancer patients. Tamoxifen, a dual antagonist and agonist of estrogen receptors, is beneficial in treating estrogen-dependent breast cancer. However, treatment with tamoxifen is less than ideal because tamoxifen's agonist behaviour enhances its unwanted estrogenic side effects. For example, tamoxifen and other compounds that agonize estrogen receptors tend to increase cancer cell production in the uterus. A better therapy for such cancers would be an anti-estrogen compound that has negligible or nonexistent agonist properties.

Although estrogen can be beneficial for treating pathologies such as bone loss, increased lipid levels, and cancer, long-term estrogen therapy has been implicated in a variety of disorders, including an increase in the risk of uterine and endometrial cancers. These and other side effects of estrogen replacement therapy are not acceptable to many women, thus limiting its use.

In addition to post-menopausal women, men suffering from prostatic cancer can also benefit from anti-estrogen compounds. Prostatic cancer is often endocrine-sensitive; androgen stimulation fosters tumor growth, while androgen suppression retards tumor growth. The administration of estrogen is helpful in the treatment and control of prostatic cancer because estrogen administration lowers the level of gonadotropin and, consequently, androgen levels. The estrogen receptor has been found to have two forms: ER□ and ERβ. Ligands bind differently to these two forms, and each form has different tissue specificity to binding ligands. Thus, it is possible to have compounds that are selective for ER□ or ERβ, and therefore confer a degree of tissue specificity to a particular ligand.

What is needed in the art are compounds that can produce the same positive responses as natural estrogens without the negative side effects. Also needed are estrogen-like compounds that exert selective effects on different tissues of the body.

The invention is related to new unnatural steroid estrogen analogues, namely, to 6-oxa-8α-steroid estrogens and a new method for preparing these compounds.

The compounds of the instant invention have anti-inflammatory activity, antiproliferative activity, osteoprotective activity and/or cholesterol lowering activity. In addition, the newly synthesized analogues could be used as precursor for sulphatase estrone inhibitors, and as estrogen receptor modulators may be useful for the treatment of a variety of conditions related to estrogen functioning.

### SUMMARY OF THE INVENTION

The present invention relates to compounds of the following chemical formula:

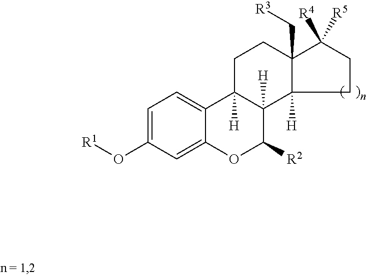

R¹═H, CH₃, Ac; R²═H, CH₃; R³═H, CH₃, CH₃CH₂; R⁴═OAc; R⁵═H, R⁴+R⁵═O

The present invention also relates to pharmaceutical compositions comprising the compounds of the present invention and a pharmaceutically acceptable carrier.

The present invention also relates to methods for making the pharmaceutical compositions of the present invention.

The present invention is also related to processes and intermediates useful for making the compounds and pharmaceutical compositions of the present invention.

The present invention also relates to methods for eliciting an estrogen receptor modulating effect in a mammal in need thereof by administering the compounds and pharmaceutical compositions of the present invention.

The present invention also relates to methods for eliciting an estrogen receptor antagonizing effect in a mammal/patient in need thereof by administering the compounds and pharmaceutical compositions of the present invention. The estrogen receptor antagonizing effect can be either an ERα antagonizing effect, and ERβ antagonizing effect or a mixed ERα and ERβ antagonizing effect.

The present invention also relates to methods for eliciting an estrogen receptor agonizing effect in a mammal in need thereof by administering the compounds and pharmaceutical compositions of the present invention. The estrogen receptor agonizing effect can be either an ERα agonizing effect, and ERβ agonizing effect or a mixed ERα and ERβ agonizing effect.

The present invention also relates to methods for treating or preventing disorders related to estrogen functioning, metabolic syndrome effecting bones, cartilages, or body weight, cancer of the breast, uterus or prostate, inflammatory diseases such as rheumatoid arthritis, colitis ulcerosa, morbus crohn, septicemia or endometriosis, cardiovascular disease, impairment of cognitive function, cerebral degenerative disorders, restenosis, gynacomastia, vascular smooth musle cell proliferation, and incontinence in a mammal in need thereof by administering the compounds and pharmaceutical compositions of the present invention.

The present invention also relates to processes for preparing of 6-oxa-8α-steroid estrogen analogues.

### DETAILED DESCRIPTION OF THE INVENTION

The present invention relates to compounds useful as estrogen receptor modulators. Compounds of the present invention are described by the following chemical formula:

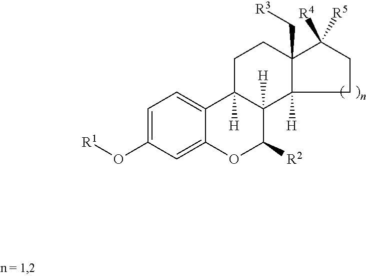

R¹═H, CH₃, Ac; R²═H, CH₃; R³═H, CH₃, CH₃CH₂; R⁴═OAc; R⁵═H, R⁴+R⁵═O

An embodiment of the invention is a method of eliciting an estrogen receptor modulating effect in a mammal in need thereof, comprising administering to the mammal a therapeutically effective amount of any of the compounds described above or any of a pharmaceutical compositions thereof. Representative compounds of the present invention typically display submicromolar affinity for alpha and/or beta estrogen receptors. Compounds of this invention are therefore useful in treating mammals suffering from disorders related to estrogen functioning. Pharmacologically effective amounts of the compound, including the pharmaceutically effective salts thereof, are administered to the mammal, to treat disorders related to estrogen functioning.

One class of the embodiment is the method wherein the estrogen receptor modulating effect is an antagonizing effect. A subclass of the embodiment is the method wherein the estrogen receptor is an ERα receptor. A second subclass of the embodiment is the method wherein the estrogen receptor is an ERβ receptor. A third subclass of the embodiment is the method wherein the estrogen receptor modulating effect is a mixed ERα and ERβ receptor antagonizing effect.

A second class of the embodiment is the method wherein the estrogen receptor modulating effect is an agonizing effect. A subclass of the embodiment is the method wherein the estrogen receptor is an ERα receptor. A second subclass of the embodiment is the method wherein the estrogen receptor is an ERβ receptor. A third subclass of the embodiment is the method wherein the estrogen receptor modulating effect is a mixed ERα and ERβ receptor agonizing effect.

Another embodiment of the invention is a method of treating or preventing post-menopausal osteoporosis, increased levels of LDL cholesterol, cardiovascular diseases, impairment of cognitive function, cerebral degeneration disorders, endometriosis and other types of inflammation, the metabolic syndrome, and cancer, in particular of the breast, uterus and prostate in a mammal in need thereof by administering to the mammal a therapeutically effective amount of any of the compounds described above or any of a pharmaceutical compositions thereof.

The compounds of the present invention can be used in combination with other agents useful for treating estrogen-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the term “composition” is intended to encompass a product comprising the specified ingredients in the specified amounts, as well as any product which results, directly or indirectly, from combination of the specified ingredients in the specified amounts. The compounds of the present invention can be administered in such oral dosage forms as tablets, capsules (each of which includes sustained release or timed release formulations), pills, powders, granules, elixirs, tinctures, suspensions, syrups and emulsions. Likewise, they may also be administered in intravenous (bolus or infusion), intraperitoneal, topical (e.g., ocular eye drop), subcutaneous, intramuscular or transdermal (e.g., patch) form, all using forms well known to those of ordinary skill in the pharmaceutical arts.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient.

Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily.

Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regimen.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and colouring agents can also be incorporated into the mixture. Suitable binders include starch, gelatine, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetats, polydihydropyrans, polycyanoacrylates and crosslinked or amphipathic block copolymers of hydrogels. Compounds of the present invention may be also delivered using multifunctional nanoparticles such as described in WO 2007/093451 [10].

The term “patient” is used herein, and refers to a mammal, including, but not limited to, primates, including simians and humans.

The term “prevention” in the context of the present invention means that the effects of a disease state or a disease causative agent have been obviated due to administration of an agent, such as those disclosed herein. A similar term in this context is “prophylaxis.” Moreover, the term “prophylactically effective amount” relates to the amount of a composition of the invention which is required to prevent the diseases.

As used herein, the terms “treatment”, “treating” and the like, refer to obtaining a desired pharmacologic and/or physiologic effect. The effect may be prophylactic in terms of completely or partially preventing one or more of said disease or a symptom thereof and/or may be therapeutic in terms of a partial or a complete cure for a disease and/or adverse affect attributable to the disease. “Treatment” as used herein, covers any treatment of a disease in a mammal, particularly in a human, and includes: (a) preventing the disease from occurring in a subject which may have had contact with a pathogen; (b) inhibiting the disease, i.e., arresting its development; and (c) relieving the disease, i.e., causing regression of the disease.

However, “therapeutic” and “prophylactic” treatments are to be considered in their broadest context. The term “therapeutic” does not necessarily imply that a subject is treated until total recovery. Similarly, “prophylactic” does not necessarily mean that the subject will not eventually develop symptoms that are associated with the diseases disclosed in the context of the present invention.

Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The term side effect as used herein refers to an unwanted, negative consequence associated with the administration of the pharmaceutical compounds mentioned elsewhere in this description. “Side effect” is thereby used synonymously with the term “adverse drug reaction”, whereas positive side effects are not included in the meaning of the term.

As used herein, “carrier” includes any and all solvents, dispersion media, vehicles, coatings, diluents, antibacterial and antifungal agents, isotonic and absorption delaying agents, buffers, carrier Solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Synthesis:

The present invention also relates to processes for preparing of 6-oxa-8α-steroid estrogen analogues, which are suitable for large-scale production. The synthesis was achieved by using Pd/C catalyst in THF for hydrogenation under atmospheric pressure. This hydrogenation catalyst-solvent system was effectively used for 6-oxaestra-1,3,5(10),8,14-pentaenes. Substrates for catalytic hydrogenation were synthesized in accordance with Torgov-Ananchenko scheme [3-5].

There are two well-known large scale procedures for the synthesis of 6-oxa-8a-steroid estrogens: catalytic hydrogenation of 6-oxaestra-1,3,5(10),8,14-pentaenes catalyzed by Pd/C [1] (prototype) and catalyzed by Ni/Ra [2]. The main disadvantage of the first approach is low stereoselectivity of the reaction. Thus, racemic 6-oxa-8a-estrone methyl ether is obtained from the corresponding estrapentaene with 15% yield, and 18-methyl-6-oxa-8α-estrone methyl ether—13% (in this case one more disadvantage is multi-step scheme of synthesis) [1]. The disadvantage of the second method is a need of using high pressure and also a need of using high purity benzene, thus making the cost of the target steroid very expensive. In addition, the second method also has disadvantages of having one additional step, the oxidation of hydrogenation products.

The novel compounds of the present invention can be prepared according to the procedure of the following schemes and examples, using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds.

All temperatures are degrees Celsius unless otherwise noted. 10% Pd/C, solvents and reagents (methanol, chloroform, tetrahydrofuran, acetic anhydride, acetic acid, pyridine and HCl etc.) were purchased from Acros Organics and were used without purification. Ursolic acid was separated from Arcostaphylos uva ursi (L.) and Sorbus aucuparia L.

Melting points were determinate on a Boestius melting point apparatus and were uncorrected. NMR spectra were obtained with a Bruker DPX-300 spectrometer (300 MHz for ¹H NMR spectra and 75 MHz for ¹³C NMR spectra). Elemental analyses were performed on Hewlett Packard 185B apparatus. TLC analyses were done on Silufol (Kavalier) and Alugram (Machereynagel) plates.

### Example 1

### 6-Oxa-8α-estrone methyl ether (II)

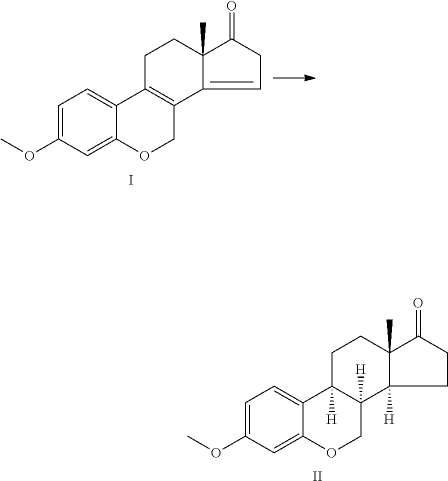

Compound II was synthesized using 10% Pd/C (300 mg) added to the solution of 3-methoxy-6-oxaestra-1,3,5(10),8,14-pentaen-17-one I (1 g) in 50 ml THF. Hydrogenation progress was monitored by UV measurements. The reaction was stopped after the characteristic aromatic wavelengths disappeared. The catalyst was then filtered and washed using THF (50 ml). Organic layers were combined; the solvent was removed in vacuum. The residue was crystallized from the mixture CHCl₃-MeOH.

The yield of the target steroid II is 64% (0.65 g), mp 149-150° C.

NMR ¹H in CDCl₃ (δ, ppm) revealed the following results: 0.93 s (3H, C¹³—CH₃), 1.43 (1H, C^{12α}—H), 1.68 (1H, C^{11β}—H), 1.84 (1H, C^{12β}—H), 1.90 (2H, C^{15α}—H and C^{15β}—H), 1.96 (1H, C^{14α}—H), 2.00 (1H, C^{11α}—H), 2.18 (1H, C^{16α}—H), 2.45 (1H, C^{16β}—H), 2.54 (1H, C^{8α}—H), 2.60 (1H, C^{9α}—H), 3.75 s (3H, O—CH₃), 4.07 (1H, C^{7β}—H), 4.22 (1H, C^{7α}—H), 6.38 (1H, C⁴—H), 6.49 (1H, C²—H), 6.99 (1H, C¹—H).

We obtained the following yields in %: C 75.46; H 7.79. C₁₈H₂₂O₃. Calcd, %: C 75.50; H 7.74.

The obtained compound is useful for the synthesis of other derivatives, such as shown in example 2.

### Example 2

### 6-Oxa-8α-estrone acetate (IV)

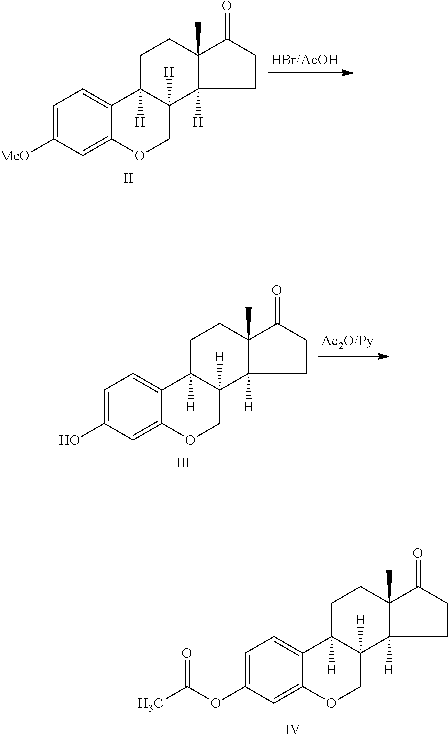

Compound IV was synthesized using a solution of 3-methoxy-6-oxa-8α-estra-1,3,5(10)-trien-17-one II (573 mg) in HBr and AcOH (20 ml, 3/7, v/v) under reflux for 2 h at 70° C. The reaction mixture was poured into water; the precipitate was filtered, rinsed with water until neutral pH. Then the product was dried on air.

The yield of product after hydrolysis was 440 mg (80.5%). The compound was used in the next stage synthesis without any additional purification.

The above compound was dissolved in 10 ml pyridine/acetic anhydride mixture (1:9 by volume), kept at 100° C. for 2.5 hours, and then left overnight at room temperature. The precipitate was filtered, washed with hexanes, and dried in vacuum.

The final yield of the target steroid IV was 230 mg (37%), mp 135-138° C.

NMR ¹H in CDCl₃ (δ, ppm) revealed the following results: 0.95 s (3H, C¹⁸—H), 1.46 td (1H, J₁=3.6 Hz, J₂=13 Hz, C¹²—H^{α}), 1.69-2.04 m (6H, C¹¹—H^{β}, C¹²—H^{β}, C¹⁵—H^{α}, C¹⁵—H^{β}, C¹⁶—H^{β}), 2.29 s (3H, C³—OCH₃), 2.14-2.34 m (1H, C¹⁴—H), 2.24 d (1H, J=8 Hz, C¹⁶—H^{α}), 2.55 m (1H, C⁸—H), 2.66 m (1H, C⁹—H), 4.09 t (1H, J=11 Hz, C⁷—H^{β}), 4.25 dd (1H, J₁=2 Hz, J₂=8 Hz, C⁷—H^{α}), 6.58 d (1H, J=2 Hz, C⁴—H), 6.64 dd (1H, J₁=2 Hz, J₂=8 Hz, C²—H), 7.10 d (1H, J=8 Hz, C¹—H).

NMR ¹³C in CDCl₃ (δ, ppm) revealed the following results: 16.95 (C¹⁸), 21.49 (C¹⁵), 21.80 (CH₃C=O), 28.27 (C¹¹), 32.08 (C¹²), 36.01 (C¹⁶), 37.27 (C⁹), 37.59 (C⁸), 46.92 (C¹⁴), 47.07 (C¹³), 64.79 (C⁷), 110.55 (C¹), 114.30 (C²), 124.88 (C¹⁰), 130.61 (C⁴), 150.03 (C⁵), 155.37 (C³), 169.89 (Ac), 219.74 (C¹⁷═O).

We obtained the following yields in %: C 72.49; H 7.09. C₁₉H₂₂O₄. Calcd., %: C 72.59; H 7.05.

### Example 3

### 6-Oxa-8α-estradiol diacetate (VI)

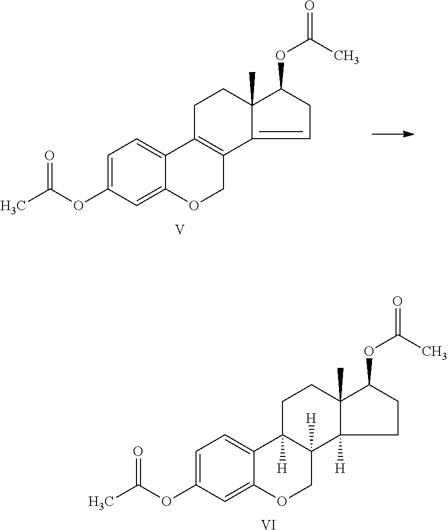

Compound VI was synthesized using 10% Pd on carbon (200 mg) added to the solution of 3,17β-diacetoxy-6-oxaestra-1,3,5(10),8,14-pentaene V (1 g) in THF (50 ml). The hydrogenation was carried out under the conditions described in the example 1. The catalyst was filtered, and washed using THF (10 ml). The solvent was removed in vacuum; the residue was crystallized from MeOH.

The yield of the target compound was 0.51 g (50%), mp 158-160° C.

NMR ¹H in CDCl₃ (δ, ppm) revealed the following results: 7.07, 1H, d, J=8.0 Hz (H—C¹); 6.61, 1H, dd, J=2.2 Hz, J=8.0 Hz (H—C²); 6.54, 1H, d, J=2.2 Hz (H—C⁴); 4.63, 1H, t, J=8.7 Hz (H—C¹⁷); 4.25-4.15, 1H, m (Hα-C⁷); 4.1-4.0, 1H, m (Hβ-C⁷); 2.65-1.3, 11H, m (H—C⁸, H—C⁹, H₂—C¹¹, H₂—C¹², H—C¹⁴, H₂—C¹⁵, H₂—C¹⁶); 2.26, 3H, s (H₃CCOO—C³); 2.05, 3H, s (H₃CCOO—C¹⁷); 0.84, 3H, s (H₃—C18).

NMR ¹³C in CDCl₃ (δ, ppm) revealed the following results: 171.2 (C(═O)—OC¹⁷); 169.7 (C(═O)—OC³); 155.3 (C³); 149.7 (C⁵); 130.4 (C¹); 125.1 (C¹⁰); 113.8 (C²); 110.2 (C⁴); 82.2 (C¹⁷); 64.4 (C⁷); 45.5; 45.5; 41.6; 37.5; 36.9; 36.1; 28.2; 27.0; 22.4; 21.3; 13.8 (C¹⁸).

We obtained the following yields in %: C 70.26; H 7.40. C₂₁H₂₆O₅. Calcd., %: C 70.37; H 7.31.

### Example 4

### 3-Methoxy-18-methyl-6-oxa-8α-estra-1,3,5(10)-trien-17-one (VIII)

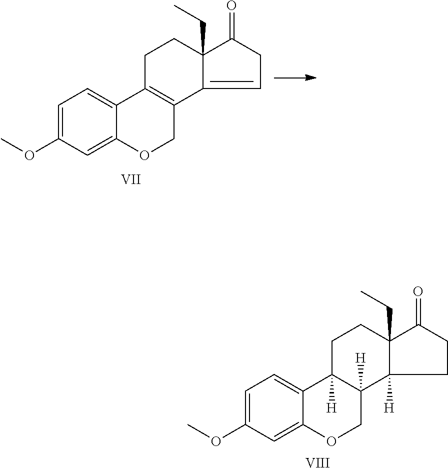

Compound VIII was synthesized using 10% Pd on carbon (100 mg) added to the solution of 3-methoxy-18-methyl-6-oxaestra-1,3,5(10),8,14-pentaene VII (1 g) in THF (40 ml). The hydrogenation was carried out under the conditions described in the example 1. The catalyst was filtered, and was washed by THF (10 ml). The solvent was removed in vacuum; the residue was crystallized from MeOH.

Mp 138-139° C.

NMR ¹H in CDCl₃ (δ, ppm) revealed the following results: 0.77 s (3H, C^{18a}—CH₃), 1.27 (1H, C^{12α}—H), 1.43 t (2H, C¹⁸—CH₃), 1.61 (1H, C^{11β}—H), 1.84 (1H, C^{12β}—H), 1.90 (2H, C^{15α}—H and C^{15β}—H), 1.96 (1H, C^{14α}—H), 2.00 (1H, C^{11α}—H), 2.18 (1H, C^{16α}—H), 2.54 (1H, C^{8α}—H), 2.43 (1H, C^{16β}—H), 2.61 (1H, C^{9α}—H), 3.76 s (3H, O—CH₃), 4.07 (1H, C^{7β}—H), 4.23 (1H, C^{7α}—H), 6.38 (1H, C⁴—H), 6.49 (1H).

MS, m/z (I, %): 300 (100, M⁺), 285 (3), 272 (3), 243 (4.5), 229 (3), 215 (3), 201 (47.5), 188 (16.5), 175 (10), 174 (7.5), 162 (77), 161 (63).

We obtained the following yields in %: C 75.79; H 8.17. C₁₉H₂₄O₃. Calcd., %: C 75.97; H 8.05.

### Example 5

### 18-Ethyl-3-methoxy-6-oxa-8α-estra-1,3,5(10)-trien-17-one (X)

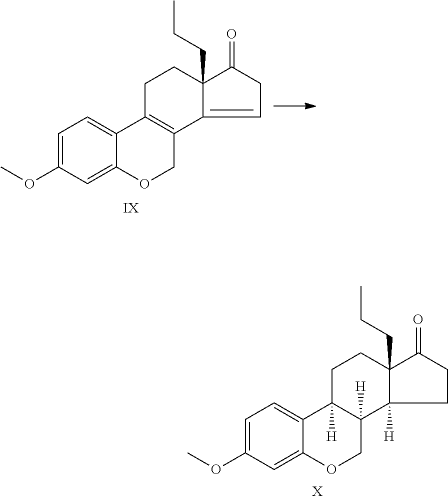

Compound X was synthesized using 10% Pd on carbon (100 mg) added to the solution of 18-ethyl-3-methoxy-6-oxaestra-1,3,5(10),8,14-pentaene IX (1 g) in THF (40 ml). The hydrogenation was carried out under the conditions described in the example 1. The catalyst was filtered, and was washed by THF (10 ml). The solvent was removed in vacuum; the residue was crystallized from MeOH. Mp 146.5-147.5° C.

MS, m/z (I, %): 314 (100, M⁺), 285 (8), 272 (3), 257 (6), 201 (39), 188 (16), 162 (66), 161 (52), 137 (15). Found, %: C 76.19, 76.34; H 8.43, 8.43. C₂₀H₂₆O₃. Calcd., %: C 76.40; H 8.34.

### Example 6

### 6-Oxa-D-homo-8α-estrone methyl ether (XII)

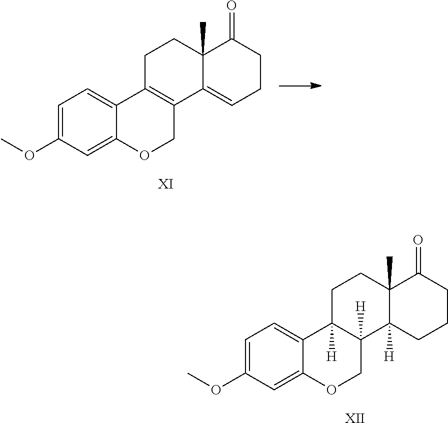

Compound XII was synthesized from the corresponding 3-methoxy-D-homo-6-oxa-estra-1,3,5(10),8,14-pentaene-17-one XI (1 g) as described in the example 1. The crystallization from the mixture CHCl₃-MeOH (1:5) gave 0.65 g (64%), mp 140.5-142° C.

MS, m/z (I, %): 300 (100), 244 (3), 229 (3), 215 (3), 201 (41), 177 (3), 175 (3), 161 (32), 147 (12).

NMR ¹H in CDCl₃ (δ, ppm) revealed the following results: 1.08 s (3H, C¹³—CH₃), 1.60 (1H, C^{15α}—H), 1.62 (1H, C^{11β}—H), 1.68 (2H, C^{12α}—H and C^{16α}—H), 1.73 (1H, C^{12β}—H), 1.83 (1H, C^{14α}—H), 1.90 (2H, C^{11α}—H and C^{15α}—H), 2.12 (1H, C^{16β}—H), 2.26 (1H, C^{8α}—H), 2.27 (1H, C^{17α}—H), 2.52 (1H, C^{9α}—H), 2.61 (1H, C^{17β}—H), 3.74 s (3H, O—CH₃), 4.09 (1H, C^{7β}—H), 4.21 (1H, C^{7α}—H), 6.35 (1H, C⁴—H), 6.47 (1H, C²—H), 6.98 (1H, C¹—H).

We obtained the following yields in %: C 75.81; H 8.21. C₁₉H₂₄O₃. Calcd., %: C 75.97; H 8.05.

### Example 7

### 7β-Methyl-D-homo-6-oxa-8α-estrone methyl ether (XIV)

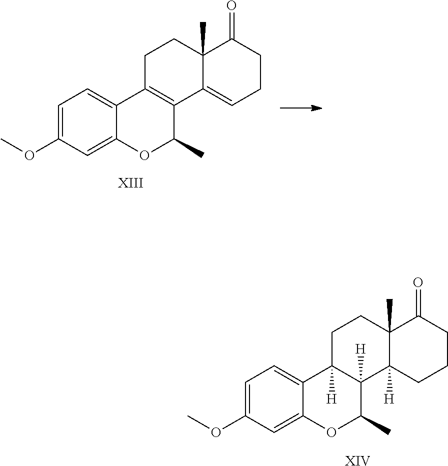

Compound XIV was synthesized using 10% Pd/C (0.3 g) added to the solution of 7β-methyl-D-homo-6-oxa-8α-estra-1,3,5(10)-pentaene XIII (1 g) in THF (100 ml). The hydrogenation was carried out under the conditions as described in example 1. The target compound (0.55 g, 54%) was obtained after the analogue separation, mp 149-151° C.

MS, m/z (I, %): 314 (100), 299 (9), 285 (6), 271 (5), 257 (5), 343 (7), 229 (5), 215 (23), 189 (11), 176 (17), 175 (22), 161 (45), 150 (22), 137 (21).

NMR ¹³C in CDCl₃ (δ, ppm) revealed the following results: 18.69, 19.47, 24.50, 26.37, 27.01, 32.02, 34.49, 37.30, 40.76, 44.80, 47.10, 55.08, 70.96, 102.34, 107.35, 118.34, 129.13, 152.87, 158.86, 214.90.

We obtained the following yields in %: C 76.29; H 8.36. C₂₀H₂₆O₃. Calcd., %: C 76.40; H 8.24.

### Example 8

### 7β-Methyl-D-homo-8α-estrone (XV)

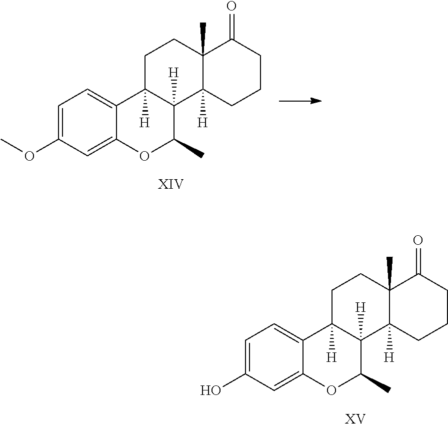

Compound XV was synthesized using a solution of 3-methoxy-7β-ethyl-D-homo-6-oxa-8α-estra-1,3,5(10)-trien-17-one XIV (103 mg) in HBr and AcOH (3 ml, 3/7, v/v) under reflux for 2 h at 70° C. The reaction mixture was poured into water; the precipitate was filtered, rinsed with water until neutral pH. Then the product was dried on air.

The yield of product after hydrolysis was 64 mg (65%). M.p. 251-253 C.

NMR ¹H in CDCl₃ (δ, ppm) revealed the following results: 1.14 s (3H, C¹³—CH₃), 1.40 d (7 Hz, 3H), 1.83-2.14 m (10H), 2.5-2.7 m (3H), 4.35-4.40 m (1H), 6.06 d (2 Hz, 1H), 6.23 dd (6 Hz, 2 Hz, 1H), 6.89 d (6 Hz, 1H), 8.79 (1H, OH).

### Example 9

### 17β-Acethoxy-3-methoxy-7β-methyl-D-homo-estra-1,3,5(10)-triene (XVI)

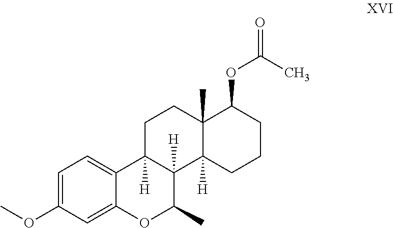

Compound XVI was obtained from steroid XIV according to standard procedures [3-5].

Mp 201-203 C.

Biological Properties:

The 6-oxa-8α-steroid estrogen analogues as prepared with the methods described under Synthesis are estrogen receptor modulators and thus possess osteoprotective and cholesterol lowering activities. These analogues also possess anti-inflammatory and antiproliferative activities. Moreover, such analogues are of great interest as pre-cursors for the manufacturing of compounds with other biological properties. Thus, sulphatase estrone inhibitor was obtained from compound XV. This inhibitor has potential for the treatment of hormone dependent breast cancer [7, 8].

The utility of the compounds of the instant invention can be readily determined by methods well known to one of ordinary skill in the art. These methods may include, but are not limited to, the following methods:

Estrogen Receptor Binding Assay;

Ovariectomized Rat Assay;

Rat Cholesterol Lowering Assay;

MCF-7 Estrogen Dependent Proliferation Assay;

Rat endometriosis model.

Examples for biological properties of 6-oxa-8a-steroid estrogen analogues are given in the following sections and Tables.

### Example 1

### Osteoprotective and Cholesterol Lowering Properties

6-oxa-8α-steroid estrogen analogues possess osteoprotective and cholesterol lowering activities. The biological properties of the 6-oxa-8α-steroid analogues IV, XII, XIV and XV in this regard are summarized in Tables 1-5 as examples.

The investigation of biological properties of steroids was carried out on sham operated and ovariectomized rats (Sprague Dawley) under the following conditions: compounds IV, XII, XIV and XV were given in olive oil, daily for 35 days [6].

TABLE 1
Properties of 6-oxa-8α-estrone acetate (compound IV) in ovariectomised rats on the
uterus, the femur, and serum cholesterol levels. 17α-Ethynylestradiol (EE) was used as a
control treatment.

| | | Uterus | Femur weight index | |
|---|---|---|---|---|
| | Change of | weight index, | (ash femur weight/ | Serum |
| Experimental group | body weight, | (mg/100 g | wet femur weigh, | cholesterol, |
| (n = 20 rats) | (g) | BW) | mg/mgt) | (mg/dl) |
| Sham-operated | 29.5 ± 3.2* | 154.2 ± 14.0* | 0.4316 ± 0.0070* | 53.7 ± 1.3* |
| Ovariectomized | 62.0 ± 5.2 | 32.0 ± 3.0 | 0.4047 ± 0.0050 | 69.4 ± 1.4 |
| Ovariectomized, treated | 23.0 ± 4.3* | 154.0 ± 7.8* | 0.4228 ± 0.0054* | 30.0 ± 1.7* |
| with EE, (0.1 mg/kg BW) | | | | |
| Ovariectomized, treated | 45.5 ± 3.8* | 71.8 ± 3.6* | 0.4219 ± 0.005* | 51.9 ± 1.7* |
| with compound IV, (0.5 mg/kg | | | | |
| BW) | | | | |

Abbreviations:
BW, body weight;
significance level of p < 0.05 is indicated by an asterisk (*)

TABLE 2
The effects of D-homo-8α-estrone methyl ether (compound XII) on
serum lipids and liver lipids of intact and ovariectomised rats.

| Experimental group | Dose, mg/kg, | Serum lipids | | Liver lipids | |
|---|---|---|---|---|---|
| (number of animals | intramuscular | Cholesterol, | Triglycerides, | Cholesterol, | Triglycerides, |
| in the group) | injection | mmol/l | mmol/l | mg/g | mg/g |
| Intact (8) | Solvent | 1.78 ± 0.15 | 0.62 ± 0.03 | 2.15 ± 0.23 | 9.1 ± 1.4 |
| Ovariectomised (8) | Solvent | 2.09 ± 0.07 | 0.59 ± 0.03 | 2.68 ± 0.08 | 7.59 ± 0.96 |
| Ovariectomised, | 0.1 | 1.68 ± 0.06* | 0.42 ± 0.05* | 2.69 ± 0.06 | 5.21 ± 0.49 |
| treated with | | | | | |
| compound XII (12) | | | | | |

TABLE 3
The effects of D-homo-8α-estrone methyl ether (compound XII) on serum lipids
and liver lipids of intact and ovariectomised rats.

| Experimental group | Dose, mg/kg, | Serum lipids | | Liver lipids | |
|---|---|---|---|---|---|
| (number of animals | intramuscular | Cholesterol, | Triglycerides, | Cholesterol, | Triglycerides, |
| in the group) | injection | mmol/l | mmol/l | mg/g | mg/g |
| Intact (8) | Solvent | 1.68 ± 0.06* | — | 4.38 ± 0.41 | 5.62 ± 0.31 |
| Ovariectomised (7) | Solvent | 2.02 ± 0.11 | 0.87 ± 0.11 | 3.93 ± 0.12 | 5.30 ± 0.80 |
| Ovariectomised, | 0.2 | 1.04 ± 0.12* | 0.85 ± 0.05 | 2.69 ± 0.06* | 3.72 ± 0.51 |
| treated with | | | | | |
| compound XII (8) | | | | | |
| Ovariectomised, | 0.5 | 0.85 ± 0.08* | 0.94 ± 0.05 | 2.31 ± 0.06* | 3.17 ± 0.32* |
| treated with | | | | | |
| compound XII (7) | | | | | |

TABLE 4
The effects of D-homo-8α-estrone methyl ether (compound XII) on serum lipids
and liver lipids of intact and ovariectomised rats.

| Experimental group | Dose, | Serum lipids | | Liver lipids | |
|---|---|---|---|---|---|
| (number of animals in | mg/kg, | Cholesterol, | Triglycerides, | Cholesterol, | Triglycerides, |
| the group) | per os | mmol/l | mmol/l | mg/g | mg/g |
| Intact (8) | Solvent | 1.35 ± 0.07* | 1.22 ± 0.07* | 2.89 ± 0.21 | 9.6 ± 0.5 |
| Ovariectomised (7) | Solvent | 1.71 ± 0.06 | 0.81 ± 0.09 | 2.55 ± 0.13 | 8.0 ± 08 |
| Ovariectomised, treated | 0.2 | 0.99 ± 0.08* | 1.45 ± 0.14* | 3.45 ± 0.15* | 7.1 ± 0.6 |
| with compound XII (8) | | | | | |
| Ovariectomised, treated | 0.2 | 0.90 ± 0.08* | 1.04 ± 0.07 | 2.82 ± 0.12 | 7.3 ± 0.2 |
| with compound XII and | | | | | |
| ursolic acid (8) | | | | | |

Typical estrogen analogues, that have uterotropic effect, usually possess also a hypertriglyceridemic activity. This unfavorable effect could be partially depleted under the action of ursolic acid (see formula below).

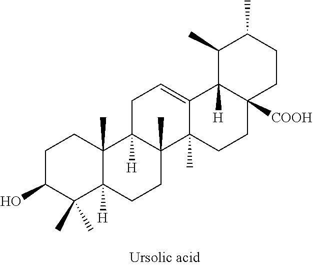

TABLE 5
Properties of 7β-methyl-D-homo-8α-estrone methyl ether (compound XIV) and 7β-
methyl-D-homo-8α-estrone (compound XV) in ovariectomised rats on the uterus, the femur,
and serum cholesterol and triglyceride levels. 17α-Ethynylestradiol (EE) was used as a
control.

| | | | Femur | | |
|---|---|---|---|---|---|
| | | Uterus | weight index | | |
| | Change | weight | (ash femur | | |
| | of body | index, | weight/wet | Serum | Serum |
| Experimental group | weight, | (mg/100 g | femur | cholesterol, | triglycerides, |
| (n = 20 rats) | (g) | BW) | weight) | (mg/dl) | (mg/dl) |
| Sham-operated | 29 ± 3* | 200 ± 12* | 0.435 ± 0.005* | 50.4 ± 2.9* | 85.4 ± 5.9* |
| Ovariectomized | 54 ± 4 | 32 ± 3 | 0.400 ± 0.003 | 69.3 ± 3.2 | 69.2 ± 5.4 |
| Ovariectomized, | 3 ± 4* | 164 ± 11* | 0.436 ± 0.004* | 45.5 ± 2.1* | 129.8 ± 14.5* |
| treated with EE (0.05 mg/kg | | | | | |
| of body weight) | | | | | |
| Ovariectomized, | 51 ± 4 | 34 ± 2* | 0.392 ± 0.004 | 54.5 ± 2.8* | 62.2 ± 5.2 |
| treated with compound | | | | | |
| XIV (5 mg/kg of body | | | | | |
| weight) | | | | | |
| Ovariectomized, | 48 ± 4 | 33 ± 3* | 0.390 ± 0.004 | 52.2 ± 2.6* | 59.6.2 ± 4.8 |
| treated with compound | | | | | |
| XV (5 mg/kg of body | | | | | |
| weight) | | | | | |

Abbreviations:
BW, body weight;
significance level of p < 0.05 is indicated by an asterisk (*)

In contrast to other analogues, the compound XV does not influence mass and number of cells in spleen, the content of antibody-forming cells, or the mass and number of cells in the thymus of female mouse-hybrids F₁CBAxC₅₇BI₆, under per os single-dosing administration (5 mg/kg of BW). Method is described in the article [9].

### Example 2

### Anti-Inflammatory Properties

6-oxa-8α-steroid estrogen analogues also possess anti-inflammatory activity. Even under single-dosing administration they possess antioxidant action. This is additional advantage in comparison with natural analogues, which possess anti-oxidant properties only under the presence of free hydroxyl group at C3. In this regard the antioxidant action of 17β-acethoxy-3-methoxy-7β-methyl-D-homo-6-oxa-estra-1,3,5(10)-triene (compound XVI) is given in table 6.

The experimental conditions: compound XVI was given per os in olive oil as a single dose of 5 mg per 100 g of body weight the day before euthanasia. Steroid solutions had a concentration of 5 mg in 0.3 ml olive oil. Control group of animals were treated using olive oil without steroids.

TABLE 6
Properties of 17β-acethoxy-3-methoxy-7β-methyl-D-homo-estra-1,3,5(10)-triene
(compound XVI) in ovariectomised rats on lipid peroxidation parameters measured in brain
tissue. Control rats received olive oil without compounds.

| | | | | | |
|---|---|---|---|---|---|
| | | | Triene | | |
| | Schiff bases, | Diene | conjugates, | | Malonic |
| Experimental | conventional | conjugates, | conventional | | dialdehyde, |
| group | units/mg of | nmol/mg of | units/mg of | Klein | nmol/mg of |
| (n = 20 rats) | phospholipids | phospholipids | phospholipids | coefficient | phospholipids |

Brain

| | | | | | |
|---|---|---|---|---|---|
| Control | 129 ± 10 | 12.2 ± 0.7 | 0.209 ± 0.022 | 0.70 ± 0.01 | 1.65 ± 0.14 |
| Compound | 96 ± 6 | 9.5 ± 1.0 | 0.152 ± 0.022 | 0.71 ± 0.05 | 1.80 ± 0.18 |
| XVI | P < 0.05 | P < 0.05 | P < 0.05 | P > 0.05 | P > 0.05 |

P - Student' coefficient.

Schiff bases contain was calculated in conventional units according to fluorometric data; triene conjugates—in conventional units—according to the spectrophotometer data under wave-length 274 nm. Klein coefficient is the ration of the spectrophotometer data under wave-length 232 nm to the data under wave-length 215 nm; this index shows the rate of lipid oxidation.

### LITERATURE

[1] Pat. 1069845. Brit., cl c07d. 8-iso-6-oxasteroids and D-homo-8-iso-6-oxasteroids. /Hughes G. A., Smith H., 1967.

[2] Patent RU 2057140 (1996) (cl. C07J 73/00). Method for obtaining of 6-oxa-8-isoanalogues of steroid estrogens. A. G. Shavva, I. I. Eliseev, Sh. N. Abusalimov and et. al.

[3] Torgov I. V. Synthesis of steroid hormones. Izv. AN SSSR, Ser. Khim. 1982, 2, 299-317.

[4] O. Dann, K-W. Hagedorn, H. Hofmann. Synthese von 7α-Methyl-6-oxa-ostron. Chem. Ber. 1971, 104, 3313-3328.

[5] Sh. N. Abusalimov, S. K. Nikol'skaja, G. L. Starova, S. I. Selivanov, A. G. Shavva. Synthesis of 6-oxa-estra-1,3,5(10),8,14-pentaenes. Russ. J. Org. Chem. 2006, 42(1), 50-55.

[6] V. N. Belov, V. Yu. Dudkin, E. A. Urusova, G. L. Starova, S. I. Selivanov, S. V. Nikolaev, N. D. Eschenko, S. N. Morozkina, A. G. Shavva. Synthesis, structure and biological properties of some 8α-analogues of steroid estrogens with fluorine in position 2. Russ. J. Bioorg. Chem. 2007, 42(3), 293-301.

[7] I. A. Gluzdikov. Synthesis of sulphatase estrone inhibitors. PhD thesis. St. Petersburg, 2007.

[8] Gluzdikov I. A., Purohit A., Reed M. J., Shavva A. G. Novel estrone sulphatase inhibitors. XVIII Mendeleev Congress on General and Applied Chemistry. Moscow, Sep. 23-28, 2007. Abstracts. P.

[9] A. G. Shavva, S. I. Selivanov, G. L. Starova et al. Russ. J. Bioorg. Chem. 2002, 28(3), 242-250.

[10]. Multimodal Imaging Using a Three Compartment Polymer Nanoparticles With Cell Specificity; WO 2007/093451.

1. A process for preparing 6-oxa-8α-steroid estrogen analogues
according to claim 13 or pharmaceutical compositions thereof, wherein a Pd/C catalyst in tetrahydrofuran (THF) is provided which hydrogenates a substrate under atmospheric pressure to prepare said 6-oxa-8α-steroid estrogen analogues or said pharmaceutical compositions.

2. (canceled)

3. The process of claim 1, wherein the -oxa-8α-steroid estrogen analogue is a compound of formula II (6-Oxa-8α-estrone methyl ether).

4. The process of claim 1, wherein the -oxa-8α-steroid estrogen analogue is a compound of formula IV (6-Oxa-8α-estrone acetate).

5. The process of claim 1, wherein the -oxa-8α-steroid estrogen analogue is a compound of formula VI (6-Oxa-8α-estradiol diacetate).

6. The process of claim 1, wherein the -oxa-8α-steroid estrogen analogue is compound of formula VIII (3-Methoxy-18-methyl-6-oxa-8α-estra-1,3,5 (10)-trien-17-one).

7. The process of claim 1, wherein the -oxa-8α-steroid estrogen analogue is a compound of formula X (18-Ethyl-3-methoxy-6-oxa-8α-estra-1,3,5 (10)-trien-17-one).

8. The process of claim 1, wherein the -oxa-8α-steroid estrogen analogue is a compound of formula XII (D-Homo-6-oxa-8α-estrone methyl ether).

9. The process of claim 1, wherein the -oxa-8α-steroid estrogen analogue is a compound of formula XIV (7β-Methyl-D-homo-6-oxa-8α-estrone methyl ether).

10. The process of claim 1, wherein the -oxa-8α-steroid estrogen analogue is a compound of formula XV (7β-Methyl-D-homo-6-oxa-8α-estrone).

11. A process to generate sulfatase estron inhibitors comprising providing the compound of claim 21 and generating sulfatase estron inhibitors.

12. The process of claim 1, wherein the -oxa-8α-steroid estrogen analogue is a compound of formula XVI (17β-Acethoxy-3-methoxy-7β-methyl-D-homo-6-oxa-estra-1,3,5(10)-triene).

13. Compounds of the following chemical formula: 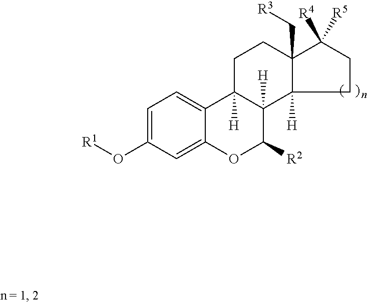
R¹═H, CH₃, Ac; R²═H, CH₃; R³═H, CH₃, CH₃CH₂; R⁴═OAc; R⁵═H, R⁴+R⁵═O.

14. Compounds according to claim 13 with general formula II:

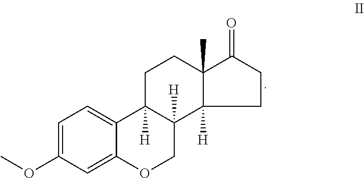

15. Compounds according to claim 13 with general formula IV:

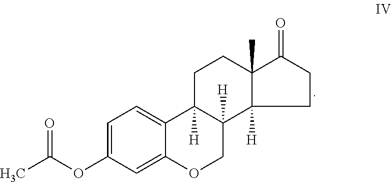

16. Compounds according to claim 13 with general formula VI:

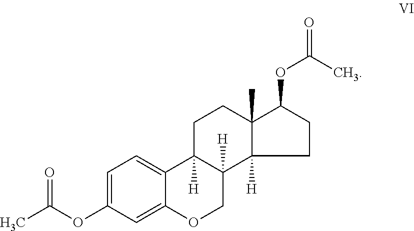

17. Compounds according to claim 13 with general formula VIII:

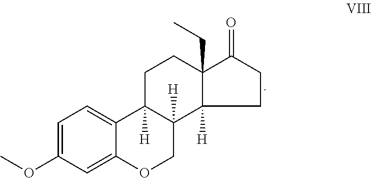

18. Compounds according to claim 13 with general formula X:

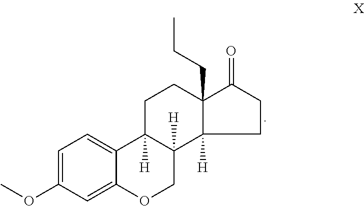

19. Compounds according to claim 13 with general formula XII:

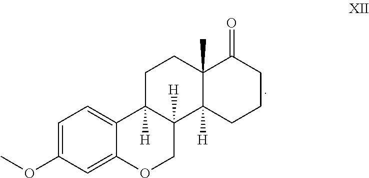

20. Compounds according to claim 13 with general formula XIV:

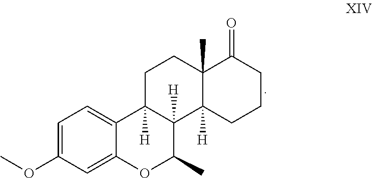

21. Compounds according to claim 13 with general formula XV:

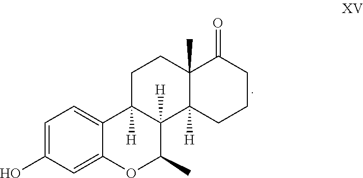

22. Compounds according to claim 13 with general formula XVI:

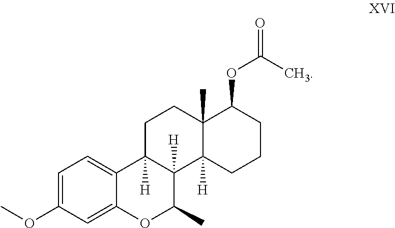

23. Compounds according to claim 13, wherein said compound is part of a medicament.

24. Method for treatment or prevention of menopausal symptoms, inflammation, dysmenorrhea and dysfunctional uterine bleeding, osteoporosis, hirsutism and/or cardiovascular disease, comprising administering to a mammal in need thereof the compound according to claim 13 in a treatment or prevention of menopausal symptoms, inflammation, dysmenorrhea and dysfunctional uterine bleeding, osteoporosis, hirsutism and/or cardiovascular disease effective amount.

25. Method for treating or preventing disorders related to estrogen functioning, metabolic syndrome effecting bones, cartilages, or body weight, cancer of the breast, uterus or prostate, inflammatory diseases such as rheumatoid arthritis, colitis ulcerosa, morbus crohn, septicemia or endometriosis, cardiovascular disease, impairment of cognitive function, cerebral degenerative disorders, restenosis, gynacomastia, vascular smooth musle cell proliferation, and/or incontinence comprising administering to a mammal in need thereof the compound of claim 13 in a disorders related to estrogen functioning, metabolic syndrome effecting bones, cartilages, or body weight, cancer of the breast, uterus or prostate, inflammatory diseases such as rheumatoid arthritis, colitis ulcerosa, morbus crohn, septicemia or endometriosis, cardiovascular disease, impairment of cognitive function, cerebral degenerative disorders, restenosis, gynacomastia, vascular smooth musle cell proliferation, and/or incontinence treating or preventing effective amount.

26. Method for treating or preventing post-menopausal osteoporosis, increased levels of LDL cholesterol, impairment of cognitive function, cerebral degeneration disorders, endometriosis, the metabolic syndrome, and/or cancer, in particular of the breast, uterus and/or prostate comprising administering to a mammal in need thereof the compound of claim 13 in a post-menopausal osteoporosis, increased levels of LDL cholesterol, impairment of cognitive function, cerebral degeneration disorders, endometriosis, the metabolic syndrome, and/or cancer, in particular of the breast, uterus and/or prostate treating or preventing effective amount.
